(19) United States
(12) Patent Application Publication
(10) Pub. No.: US 2024/0331050 A1
(43) Pub. Date: Oct. 3, 2024

(54) CONVERSATION ANALYSIS USING ARTIFICIAL INTELLIGENCE

(71) Applicant: DANIEL INOUYE, Provo, UT (US)

(72) Inventor: DANIEL INOUYE, Provo, UT (US)

(21) Appl. No.: 18/620,702

(22) Filed: Mar. 28, 2024

Related U.S. Application Data
(60) Provisional application No. 63/493,706, filed on Mar. 31, 2023.

(51) Int. Cl. G06Q 40/08; G16H 10/20

(52) U.S. Cl. CPC G06Q 40/08; G16H 10/20

(57) ABSTRACT

Apparatuses, methods, systems, and program products are disclosed for conversation analysis using artificial intelligence. An apparatus is configured to receive a recording of a conversation between a plurality of participants, process the recording using an artificial intelligence engine to identify at least one marker of the conversation, the at least one marker associated with at least one of the plurality of participants, and determine a score for the conversation or the at least one of the plurality of participants based on the at least one marker.

### CROSS-REFERENCE TO RELATED APPLICATIONS

This application claims the benefit of U.S. Provisional Patent Application No. 63/493,706 entitled “METHOD AND SYSTEM FOR EVALUATING PATIENT SATISFACTION, ASSESSING HEALTHCARE PROVIDER QUALITY OF CARE, AND IDENTIFYING RISK OF MALPRACTICE LITIGATION USING TRANSCRIPTION, CONTENT AND SENTIMENT ANALYSIS, EMOTION RECOGNITION, AND ARTIFICIAL INTELLIGENCE” and filed on Mar. 31, 2023, for Daniel Inouye, which is incorporated herein by reference in its entirety for all purposes.

### FIELD

This invention relates to artificial intelligence and more particularly to conversation analysis using artificial intelligence.

### BACKGROUND

When insurance companies calculate premiums for healthcare providers, they typically evaluate the risk associated with insuring them. This risk assessment involves a variety of factors, such as the provider's specialty, board certification, claims history, years or practice, medical school of graduation, location of practice, hours of weekly work, and whether the practitioner is performing telehealth. Insurance companies may also consider a provider's reviews as well as any malpractice claims or lawsuits that they may have been involved in. To evaluate the risk associated with a healthcare provider, insurance companies less commonly conduct site visits, review medical records and other documents, and interview the provider and their staff. This process can be time consuming and may not provide a comprehensive understanding of the provider's performance. Traditionally, patient interactions with healthcare providers have been primarily documented through handwritten notes or typed summaries. These notes and summaries are generally written from the perspective of the doctor and necessarily brief with large amounts of potentially useful data omitted.

### BRIEF SUMMARY

An apparatus, in one embodiment, is configured to receive a recording of a conversation between a plurality of participants, process the recording using an artificial intelligence engine to identify at least one marker of the conversation, the at least one marker associated with at least one of the plurality of participants, and determine a score for the conversation or the at least one of the plurality of participants based on the at least one marker.

A method, in one embodiment, includes receiving a recording of a conversation between a plurality of participants, processing the recording using an artificial intelligence engine to identify at least one marker of the conversation, the at least one marker associated with at least one of the plurality of participants, and determining a score for the conversation or the at least one of the plurality of participants based on the at least one marker.

An apparatus, in one embodiment, includes means for receiving a recording of a conversation between a plurality of participants, means for processing the recording using an artificial intelligence engine to identify at least one marker of the conversation, the at least one marker associated with at least one of the plurality of participants, and means for determining a score for the conversation or the at least one of the plurality of participants based on the at least one marker.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 illustrates one embodiment of a system in accordance with the subject matter described herein;

FIG. 2 illustrates one embodiment of an apparatus in accordance with the subject matter described herein;

FIG. 3 illustrates one embodiment of method in accordance with the subject matter described herein;

FIG. 4 illustrates one embodiment of a method in accordance with the subject matter described herein; and

FIG. 5 illustrates one embodiment of a method in accordance with the subject matter described herein.

### DETAILED DESCRIPTION

In general, the subject matter herein is directed to apparatuses, methods, program products, and systems for evaluating patient satisfaction, assessing healthcare provider quality of care, and identifying risk of potential malpractice litigation using transcription, content and sentiment analysis, emotion recognition, and artificial intelligence is disclosed. The system uses one or more devices with one or more microphones, cameras, displays, and processors which have been trained using machine learning algorithms to identify markers that vary with levels of patient satisfaction/dissatisfaction, levels in quality of care, and/or levels of risk of potential malpractice litigation.

When insurance companies calculate premiums for healthcare providers, they typically evaluate the risk associated with insuring them. This risk assessment involves a variety of factors, such as the provider's specialty, board certification, claims history, years or practice, medical school of graduation, location of practice, hours of weekly work, and whether or not the practitioner is performing telehealth. Insurance companies may also consider a provider's reviews as well as any malpractice claims or lawsuits that they may have been involved in. To evaluate the risk associated with a healthcare provider, insurance companies less commonly conduct site visits, review medical records and other documents, and interview the provider and their staff. This process can be time-consuming and may not provide a comprehensive understanding of the provider's performance. Traditionally, patient interactions with healthcare providers have been primarily documented through handwritten notes or typed summaries. These notes and summaries are generally written from the perspective of the doctor and necessarily brief with large amounts of potentially useful data omitted.

With the advent of speech-to-text transcription technologies, it has become possible to automatically transcribe and analyze the interactions between healthcare providers and patients in a more efficient and accurate manner. The development of natural language processing (NLP) and content and sentiment analysis algorithms can be used to extract valuable insights from these interactions.

The subject matter herein combines markers found using transcription, content and sentiment analysis, emotion recognition, and artificial intelligence (AI) and machine learning (ML) algorithms to identify trends and patterns in healthcare provider behavior, which correlates to patient satisfaction, quality of care, and risk of malpractice litigation. This technology has the potential to revolutionize the way healthcare providers are evaluated and could lead to significant improvements in the quality of care delivered to patients. It also can help insurance companies to assess risk and adjust premiums accordingly.

The subject matter described herein provides improvements over traditional methods for evaluating healthcare provider performance and risk of malpractice litigation, including:
1. Increased accuracy: By using speech-to-text transcription, natural language processing, and sentiment analysis algorithms, the invention can more accurately and objectively evaluate provider quality of care and patient satisfaction. This technology eliminates the potential for human bias and subjectivity in evaluating provider interactions with patients.
2. Efficiency: The use of AI and ML algorithms enables the invention to process large volumes of data quickly and efficiently, allowing for more comprehensive and frequent evaluations of healthcare providers.
3. Improved risk assessment: By combining the above data with a variety of other sources (i.e. including patient satisfaction surveys, clinical notes, and malpractice claims), the invention can provide a more comprehensive and accurate assessment of the risk associated with insuring healthcare providers.
4. Cost savings: By accurately identifying high-risk healthcare providers and providing targeted interventions, the invention can help to reduce the frequency and severity of malpractice claims, resulting in cost savings for insurance companies and healthcare organizations.
5. Quality improvement: By providing healthcare providers with feedback on their performance and areas for improvement, the invention can help to improve the overall quality of care delivered to patients, resulting in better patient outcomes and satisfaction.

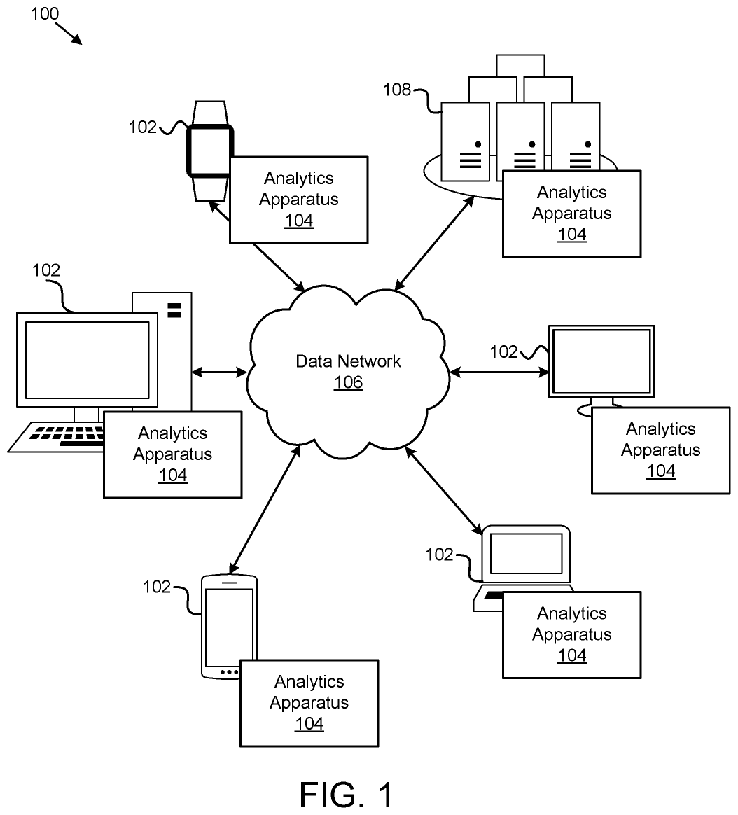

FIG. 1 is a schematic block diagram illustrating one embodiment of a system 100. In one embodiment, the system 100 includes one or more computing devices 102, one or more trust apparatuses 104, one or more data networks 106, and one or more servers 108. In certain embodiments, even though a specific number of computing devices 102, analytics apparatuses 104, data networks 106, and servers 108 are depicted in FIG. 1, one of skill in the art will recognize, in light of this disclosure, that any number of computing devices 102, analytics apparatuses 104, data networks 106, and servers 108 may be included in the system 100.

In one embodiment, the system 100 includes one or more computing devices 102. The computing devices 102 may be embodied as one or more of a desktop computer, a laptop computer, a tablet computer, a smart phone, a smart speaker (e.g., Amazon Echo®, Google Home®, Apple HomePod®), an Internet of Things device, a security system, a set-top box, a gaming console, a smart TV, a smart watch, a fitness band or other wearable activity tracking device, an optical head-mounted display (e.g., a virtual reality headset, smart glasses, head phones, or the like), a High-Definition Multimedia Interface (“HDMI”) or other electronic display dongle, a personal digital assistant, a digital camera, a video camera, or another computing device comprising a processor (e.g., a central processing unit (“CPU”), a processor core, a field programmable gate array (“FPGA”) or other programmable logic, an application specific integrated circuit (“ASIC”), a controller, a microcontroller, and/or another semiconductor integrated circuit device), a volatile memory, and/or a non-volatile storage medium, a display, a connection to a display, and/or the like.

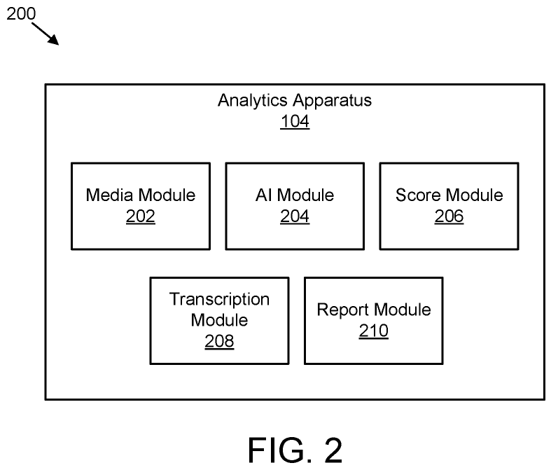

In general, in one embodiment, the analytics apparatus 104 uses audio signals to capture the interaction between different users, e.g., a healthcare provider and the patient. In one embodiment, the analytics apparatus 104 processes the captured audio using an artificial intelligence engine to identify markers within the conversation that may be associated with patient satisfaction, quality of care, and risk of malpractice litigation. In one embodiment, the analytics apparatus 104 may determine a score for the conversation that can be used to rate the conversation, the user's involved in the conversation, and/or the like. The analytics apparatus 104 is described in more detail below with reference to FIG. 2.

In certain embodiments, the analytics apparatus 104 may include a hardware device such as a secure hardware dongle or other hardware appliance device (e.g., a set-top box, a network appliance, or the like) that attaches to a device such as a head mounted display, a laptop computer, a server 108, a tablet computer, a smart phone, a security system, a network router or switch, or the like, either by a wired connection (e.g., a universal serial bus (“USB”) connection) or a wireless connection (e.g., Bluetooth®, Wi-Fi, near-field communication (“NFC”), or the like); that attaches to an electronic display device (e.g., a television or monitor using an HDMI port, a DisplayPort port, a Mini DisplayPort port, VGA port, DVI port, or the like); and/or the like. A hardware appliance of the analytics apparatus 104 may include a power interface, a wired and/or wireless network interface, a graphical interface that attaches to a display, and/or a semiconductor integrated circuit device as described below, configured to perform the functions described herein with regard to the analytics apparatus 104.

The analytics apparatus 104, in such an embodiment, may include a semiconductor integrated circuit device (e.g., one or more chips, die, or other discrete logic hardware), or the like, such as a field-programmable gate array (“FPGA”) or other programmable logic, firmware for an FPGA or other programmable logic, microcode for execution on a microcontroller, an application-specific integrated circuit (“ASIC”), a processor, a processor core, or the like. In one embodiment, the analytics apparatus 104 may be mounted on a printed circuit board with one or more electrical lines or connections (e.g., to volatile memory, a non-volatile storage medium, a network interface, a peripheral device, a graphical/display interface, or the like). The hardware appliance may include one or more pins, pads, or other electrical connections configured to send and receive data (e.g., in communication with one or more electrical lines of a printed circuit board or the like), and one or more hardware circuits and/or other electrical circuits configured to perform various functions of the analytics apparatus 104.

The semiconductor integrated circuit device or other hardware appliance of the analytics apparatus 104, in certain embodiments, includes and/or is communicatively coupled to one or more volatile memory media, which may include but is not limited to random access memory (“RAM”), dynamic RAM (“DRAM”), cache, or the like. In one embodiment, the semiconductor integrated circuit device or other hardware appliance of the analytics apparatus 104 includes and/or is communicatively coupled to one or more non-volatile memory media, which may include but is not limited to: NAND flash memory, NOR flash memory, nano random access memory (nano RAM or “NRAM”), nanocrystal wire-based memory, silicon-oxide based sub-10 nanometer process memory, graphene memory, Silicon-Oxide-Nitride-Oxide-Silicon (“SONOS”), resistive RAM (“RRAM”), programmable metallization cell (“PMC”), conductive-bridging RAM (“CBRAM”), magneto-resistive RAM (“MRAM”), dynamic RAM (“DRAM”), phase change RAM (“PRAM” or “PCM”), magnetic storage media (e.g., hard disk, tape), optical storage media, or the like.

The data network 106, in one embodiment, includes a digital communication network that transmits digital communications. The data network 106 may include a wireless network, such as a wireless cellular network, a local wireless network, such as a Wi-Fi network, a Bluetooth® network, a near-field communication (“NFC”) network, an ad hoc network, and/or the like. The data network 106 may include a wide area network (“WAN”), a storage area network (“SAN”), a local area network (“LAN”) (e.g., a home network), an optical fiber network, the internet, or other digital communication network. The data network 106 may include two or more networks. The data network 106 may include one or more servers, routers, switches, and/or other networking equipment. The data network 106 may also include one or more computer readable storage media, such as a hard disk drive, an optical drive, non-volatile memory, RAM, or the like.

In one embodiment, the data network 106 is a mesh network. As used herein, a mesh network is a local area network topology in which the infrastructure nodes (i.e. bridges, switches, and other infrastructure devices) connect directly, dynamically and non-hierarchically to as many other nodes as possible and cooperate with one another to efficiently route data to and from clients. This lack of dependency on one node allows for every node to participate in the relay of information.

The wireless connection may be a mobile telephone network. The wireless connection may also employ a Wi-Fi network based on any one of the Institute of Electrical and Electronics Engineers (“IEEE”) 802.11 standards. Alternatively, the wireless connection may be a Bluetooth® connection. In addition, the wireless connection may employ a Radio Frequency Identification (“RFID”) communication including RFID standards established by the International Organization for Standardization (“ISO”), the International Electrotechnical Commission (“IEC”), the American Society for Testing and Materials® (ASTM®), the DASH7™ Alliance, and EPCGlobal™.

Alternatively, the wireless connection may employ a ZigBee® connection based on the IEEE 802 standard. In one embodiment, the wireless connection employs a Z-Wave® connection as designed by Sigma Designs®. Alternatively, the wireless connection may employ an ANT® and/or ANT+® connection as defined by Dynastream® Innovations Inc. of Cochrane, Canada.

The wireless connection may be an infrared connection including connections conforming at least to the Infrared Physical Layer Specification (“IrPHY”) as defined by the Infrared Data Association® (“IrDA”®). Alternatively, the wireless connection may be a cellular telephone network communication. All standards and/or connection types include the latest version and revision of the standard and/or connection type as of the filing date of this application.

The one or more servers 108, in one embodiment, may be embodied as blade servers, mainframe servers, tower servers, rack servers, and/or the like. The one or more servers 108 may be configured as mail servers, web servers, application servers, FTP servers, media servers, data servers, web servers, file servers, virtual servers, and/or the like. The one or more servers 108 may be communicatively coupled (e.g., networked) over a data network 106 to one or more computing devices 102.

FIG. 2 depicts one embodiment of an apparatus 200 for conversation analysis using artificial intelligence. The apparatus 200 may include an instance of an analytics apparatus 104. The analytics apparatus 104 may include one or more of a media module 202, an AI module 204, a score module 206, a transcription module 208, and a report module 210, which are described in more detail below. In one embodiment, the apparatus 104 is a hardware device that is specially configured for capturing a conversation between the plurality of participants and performing the functions of the apparatus 104.

In one embodiment, the media module 202 is configured to receive a recording of a conversation between a plurality of participants. In one embodiment, the recording comprises an audio recording or a video recording of the conversation between a plurality of participants. In certain embodiments, at least one of the plurality of users is a health care provider and another of the plurality of users is a patient of the health care provider.

In one embodiment, the media module 202 is triggered to record a conversation in response to detecting that the users are having a conversation, e.g., in response to detecting audio or video that indicates that the users are speaking to one another. In one embodiment, the media module 202 may be part of an application (e.g., a mobile application), such as a telehealth or video conference application, may be located on a device that is dedicated to recording audio or video, such as a voice recorder, and/or the like. In one embodiment, the media module 202 may store the recording in a data store. The data store may be located locally to the media module 202, may be remotely located, e.g., in a cloud data store, and/or the like.

In one embodiment, the AI module 204 is configured to receive a recording, e.g., via an application programming interface (API) or other interface and process the recording using an AI engine to identify at least one marker of the conversation. As used herein, a marker may refer to a word, phrase, emotion, sentiment, tone, expression, gesture, and/or the like that is associated with a predefined category. In such an embodiment, the at least one marker is associated with at least one of the plurality of participants.

AI, as used herein, is broadly defined as a branch of computer science dealing in automating intelligent behavior. AI systems may be designed to use machines to emulate and simulate human intelligence and corresponding behavior. This may take many forms, including symbolic or symbol manipulation AI. AI may address analyzing abstract symbols and/or human readable symbols. AI may form abstract connections between data or other information or stimuli. AI may form logical conclusions. AI is the intelligence exhibited by machines, programs, or software. AI has been defined as the study and design of intelligent agents, in which an intelligent agent is a system that perceives its environment and takes actions that maximize its chances of success.

AI may have various attributes such as deduction, reasoning, and problem solving. AI may include knowledge representation or learning. AI systems may perform natural language processing, perception, motion detection, and information manipulation. At higher levels of abstraction, it may result in social intelligence, creativity, and general intelligence. Various approaches are employed including cybernetics and brain simulation, symbolic, sub-symbolic, and statistical, as well as integrating the approaches.

Various AI tools may be employed, either alone or in combinations. The tools may include search and optimization, logic, probabilistic methods for uncertain reasoning, classifiers and statistical learning methods, neural networks, deep feedforward neural networks, deep recurrent neural networks, deep learning, control theory and languages.

In one embodiment, the AI engine may include a generative AI engine. As used herein, generative AI is a type of AI that can create new content, such as text, images, music, audio, and videos. Generative AI systems are often used to develop synthetic data, which can be used to train machine learning models and validate mathematical models. In such an embodiment, prompts may be provided to the generative AI engine for generation of content. For example, as used herein, the AI module 204 may provide a prompt such as “transcribe this audio stream,” “identify sentiment markers within this transcription,” “rate the healthcare provider's conversational skills,” or the like.

In such an embodiment, the AI module 204 may train an AI engine on subject-specific data such as historical conversation data, sample conversation snippets with known markers or scores, and/or the like. The AI module 204 may continuously train and refine an AI engine on new conversation data, audio/video recordings, transcription analysis, e.g., using natural language processing, and/or the like. Thus, in one embodiment, the AI module 204 trains the AI engine to identify at least one marker of the conversation using marker-related data.

In one embodiment, the at least one marker comprises a communication skill marker that indicates a communication skill of at least one of the plurality of participants. As used herein, a communication skill marker may be a part of a conversation that identifies the depth of communication skills of a provider during patient appointments. Communication skill markers may include one of more of the following:
a. Greeting markers: These markers identify the extent to which a provider greets their patients.
b. Small talk markers: These markers identify the extent to which a provider engages patients with nonmedical conversation.
c. Orientation markers: These markers identify the degree to which a provider orients their patient. For example, the phrase “First, I will examine you and then you will have some tests” is a positive indication of orientation.
d. Facilitation markers: These markers identify the degree to which a provider facilitates communication. These markers often indicate that a provider is asking a patient about their opinions or checking their understanding. For example, the phrase “Go on, tell me more” is a positive indication of facilitation.
e. Provider approval markers: These markers identify the degree to which a provider expresses approval of their patient. For example, “You're doing great with your weight loss goal” is a positive indication of provider approval.
f. Provider concern markers: These markers identify the degree to which a provider expresses concern for a patient. For example, “If you are having chest pain, we should probably check your blood pressure” is a positive indication of provider concern.
g. Provider team mindset markers: These markers identify the degree to which a provider expresses a team-like rapport with the patient. For example, criticism of third parties, such as “Insurance companies can be so annoying. They really should cover the medication” is a positive indication of provider team mindset.
h. Provider listening markers: These markers identify the degree to which a provider listens to a patient.
i. Provider interruption markers: These markers identify the degree to which a provider interrupts a patient.
j. Provider attention markers: These markers identify the degree to which a provider gives a patient undivided attention.
k. Provider apology markers: These markers identify the degree to which a provider apologizes to a patient.
l. Provider dismissiveness markers: These markers identify the degree to which a provider acts dismissively towards patient or family concerns.
m. Condolences markers: These markers identify the degree to which providers offer condolences to patients after learning of tragedy.
n. Medical terminology frequency markers: These markers identify the frequency of medical terminology being used by providers in visits.
o. Medical terminology defined markers: These markers identify the frequency and extent to which medical terminology is explained to the patient when used by the provider.
p. Backchanneling markers: These markers identify the degree to which a provider gives backchannel signals in an encounter. For example, “yeah,” “okay,” “uh-huh.”
q. No further question markers: This marker identifies visits in which patients have been able to ask questions to the point where they have no additional questions.
r. Open-ended question markers: These markers identify the degree to which a provider asks open-ended questions.
s. Rushed provider markers: These markers identify the degree to which a provider comes across as rushed.

In one embodiment, the at least one marker comprises a content marker. As used herein, content markers may identify conversational content during patient appointments. Content markers are meaningful because they may be associated with a degree of patient satisfaction, quality of care, and/or risk of potential malpractice litigation. Content markers may include one of more of the following:
a. Provider utterances marker: This marker quantifies the number of utterances made by a provider in a given visit.
b. Visit duration: This marker quantifies the duration of a visit.
c. Lifestyle and social issues counseling markers: These markers identify the degree to which the provider provided counseling on lifestyle and social issues.
d. Medication and treatments counseling markers: These markers identify the degree to which the provider gave counseling on medication or other treatments.
e. Patient medical question markers: These markers identify the number and complexity of questions a patient asked about medical issues.
f. Patient psychosocial and lifestyle question markers: These markers identify the number and complexity of questions a patient asked about psychosocial and lifestyle issues.
g. Patient agreement markers: These markers identify the degree to which a patient agrees with the provider.
h. Shared decision-making markers: These markers identify the degree to which a provider has adequately informed a patient about care options, risks and benefits of options, incorporates the patient's values and priorities in the decision process, and allows the patient to participate in making decisions about the care to be provided.
i. Diagnoses markers: These markers identify the medical issues addressed in a visit.
j. Procedure markers: These markers identify the procedures planned or performed during a visit.
k. Drug prescription markers: These markers identify the medications prescribed or offered in a visit.
l. Referral markers: These markers identify the number and the type of referrals planned or made by a provider.
m. Ordered test markers: These markers identify the number and types of tests, such as laboratories or imaging studies, ordered or offered by a provider.
n. Managed expectations markers: These markers identify the degree to which a provider manages patient and family expectations.
o. Offensiveness markers: These markers identify the degree to which providers make offensive comments, including politically incorrect comments or racist language.
p. Mainstream practice markers: These markers identify the degree to which a provider acts according to mainstream practice.

In one embodiment, the at least one marker comprises a sentiment and emotion recognition marker. As used herein, sentiment and emotion recognition markers may identify provider, patient, and third-party emotions during a conversation, e.g., as part of a patient appointment. Sentiment and emotion recognition markers are meaningful in measuring the degree of patient satisfaction, quality of care, and/or risk of potential malpractice litigation. Sentiment and emotion recognition markers may include the following:
a. Provider humor markers: These markers identify the degree to which a provider uses humor and or laughter.
b. Provider-patient sentiment match markers: These markers identify the degree to which a provider's affect matches that of the patient. For example, a jovial provider and a sad patient would be a negative indication of provider-patient sentiment match.
c. Patient affect markers: These markers identify the emotions experienced by a patient in a visit.
d. Visit tone makers: These markers identify the overall tone of a physician-patient visit.
e. Provider disrespect: These markers identify disrespectful attitudes of providers towards patients or third parties.
f. Provider defensiveness markers: These markers identify defensive attitudes of providers.
g. Provider empathy markers: These markers identify a provider attitude of empathy.
h. Rapport markers: These markers identify the degree of rapport between provider and patient.
i. Provider anger markers: These markers identify the degree to which a provider expresses anger or frustration.
j. Patient anger markers: These markers identify the degree to which a patient expresses anger or frustration.
k. Provider critical attitude markers: These markers identify the degree to which a provider expresses a critical attitude towards the patient.

In one embodiment, the at least one marker comprises an identity marker. As used herein, an identity marker may indicate one or more identity characteristics of at least one of the plurality of participants. Identity markers may include the following:
a. Patient socioeconomic status markers: These markers identify the likely socioeconomic status of a patient, including their degree of education.
b. Patient reactivity markers: These markers identify reactive traits of patients.

In one embodiment, the at least one marker comprises a visual marker, e.g., captured from a video recording or image. As used herein, a visual marker may comprise a marker based on a physical feature of a user, e.g., a gesture, stance, gait, facial movement, hand movements, or the like. Visual markers may include:
a. Provider attractiveness markers: These markers identify the general physical attractiveness of a provider.
b. Provider eye contact markers: These markers identify the degree to which a provider makes eye contact with the patient.

In one embodiment, the AI module 204 may identify other markers from a plurality of conversations between participants, e.g., after several patient visits with a doctor. These markers may include:
a. Number of unique patient markers: These markers identify the number of different patients a provider sees.
b. Frequency of visits markers: These markers identify how frequently a patient is seen, as well as how often a patient is seen for a single visit.
c. Duration of relationship with patients: These markers identify how long a provider continues to treat a given patient.
d. Emergency department-like cases markers: These markers identify the degree to which a provider sees emergency-department like cases.
e. Hours of practice markers: These markers identify the number of hours a provider practices.
f. Frequency of procedures markers: These markers identify the types and frequencies of procedures performed.
g. Days away from practice markers: These markers identify the number of days away from practice a provider is taking.
h. Teaching markers: These markers identify the degree to which a provider is engaging in teaching and the frequency that trainees, physician assistants, nurse practitioners, and other collaborative providers are being used in the provider's practice.
i. Specialty markers: These markers identify the likely specialty of a provider, based on diagnoses seen and procedures performed.
j. Relative Value Unit (RVU) markers: These markers identify the number of RVUs a provider performs.

In one embodiment, the AI module 204 combines the foregoing markers with traditional risk quantification markers, such as patient satisfaction surveys, clinical notes, and malpractice claims. Specific markers that have an impact on patient satisfaction, quality of care, and the risk of malpractice litigation can then be given to providers to identify areas in which they can improve. The markers may be weighted to measure the distribution of patient satisfaction, distribution of quality of care, and the risk of malpractice litigation.

In such an embodiment, the AI module 204 sets weighting factors for each of the at least one markers of the conversation. As used herein, the weighting factors may define an importance of each of the at least one markers, and the score for the conversation, described below, may be determined as a function of the weighting factors. For instance, an entire group or category of markers, such as such as communication skill markers, may be weighted more or less important than other groups or categories of markers, or individual markers, such as offensiveness markers, may be weighted more or less important that other markers, and/or the like.

In one embodiment, the score module 206 is configured to determine a score for the conversation, for at least one of the plurality of participants of the conversation, and/or the like, based on the at least one marker. As used herein, the score module 206 may generate a score, ranking, grade, level, and/or other indicator of a conversation between participants. For example, the score module 206 may analyze each of the markers that are identified in a conversation between a doctor and a patient and determine a score for the doctor. The score may describe how effective the doctor is at communicating ideas, at listening, at answering questions, and/or the like. The score may be an overall score for the conversation, e.g., based on individual scores for each of the markers identified in the conversation, may be an average of the marker scores, may be determined for categories of markers, may be broken down into scores for each individual marker, and/or the like.

For example, each marker may be worth 1 or −1 points, and the score module 206 may determine an overall score for the conversation by aggregating the point totals for each of the identified markers (e.g., positive markers such as condolence markers may be worth 1 point while negative marks such as offensiveness markers may be worth −1 points; markers that are not identified may be assigned 0 points). Further, if the AI module 204 weighs the markers, the point values for the markers may be worth even more or less, e.g., −10 points for offensiveness markers, 5 points for provider apology markers, or the like.

In one embodiment, the score module 206 may map the score to a risk factor associated with the health care provider. As used herein, the risk factor may indicate a level of risk associated with the health care provider. For instance, the risk factor may indicate whether the health care provider is at a higher risk of being sued, of having a malpractice claim, of losing patients, and/or the like.

In one embodiment, the score module 206 may map the score to a performance level associated with the health care provider. As used herein, the performance level may indicate an effectiveness of the health care provider. For instance, the performance level may indicate whether the health care provider is accurate in their answers or diagnoses, speaks clearly, is confident, puts patients at ease, treats patients with respect, and/or the like.

In one embodiment, the score for the conversation may be indicative of a relationship between the health care provider and the patient. The score may indicate a mood of the conversation, e.g., positive, light, happy, negative, confrontational, or the like; whether the conversation is smooth or whether there are awkward pauses, long periods of quiet, confusion, laughter, and/or the like.

In one embodiment, the score module 206 may determine the score using data from at least one external data source associated with at least one of the plurality of participants. For instance, the score module 206 may further reference or incorporate survey data, note data, legal data, and/or the like to determine a risk, performance, and/or likeability of the health care professional.

In one embodiment, the transcription module 208 is configured to transcribe the recording of the conversation to text and provide the text to the AI engine for processing, e.g., via an API or other interface. The transcription module 208, for instance, may process an audio stream, track, file, or the like, e.g., from an audio or video recording, and may transcribe the audio to text.

In one embodiment, the transcription module 208 may analyze the text using natural language processing to identify and remove sensitive information such as personally identifiable information (PII), personal health information (PHI), payment card industry (PCI) information, and/or the like. In other words, the transcription module 208 may anonymize the data so that a patient's personal information is not exposed or stored to unauthorized users.

In one embodiment, the transcription module 208 generates the transcription of the audio recording without storing the transcription in persistent or nonvolatile storage prior to or after providing the transcription to the AI module 204 for processing. In this manner, the transcription data is not persistently stored to protect the user's data.

In one embodiment, the report module 210 is configured to generate a report comprising the individual scores for each of the at least one markers of the conversation for a participant of the conversation. For instance, the report module 210 may generate a report for a doctor based on one or more of the doctor's conversations with patients. In such an embodiment, the report may outline, describe, or explain the markers that were identified, the scores of the markers for the doctor and the conversation, a risk analysis, a performance analysis, a likeability analysis, and/or the like.

The report module 210 may further provide the report to third parties. In a healthcare example, a doctor's report may be provided to hospital administrators, insurance companies, and/or other stakeholders to evaluate the doctor's performance, risk, and likeability and identify areas where the doctor can improve, areas where the hospital can improve, specific areas where trainings can be provided and will be effective, and/or the like.

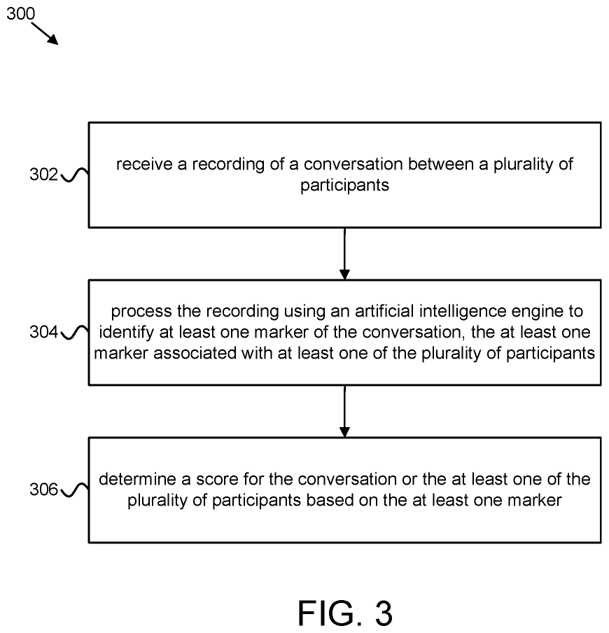

FIG. 3 depicts one embodiment of a method 300 for conversation analysis using artificial intelligence. In one embodiment, the method 300 may be performed by an information handling device 102, an analytics apparatus 104, a media module 202, an AI module 204, and/or a score module 206.

In one embodiment, the method 300 begins and receives 302 a recording of a conversation between a plurality of participants. In one embodiment, the method 300 processes 304 the recording using an artificial intelligence engine to identify at least one marker of the conversation, the at least one marker associated with at least one of the plurality of participants. In one embodiment, the method 300 determines 306 a score for the conversation or the at least one of the plurality of participants based on the at least one marker, and the method 300 ends.

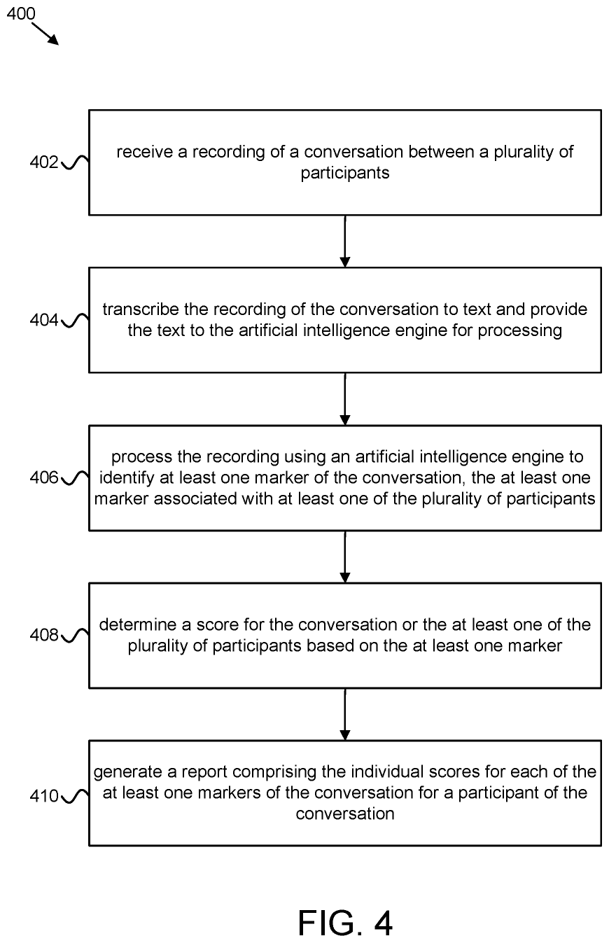

FIG. 4 depicts one embodiment of a method 400 for conversation analysis using artificial intelligence. In one embodiment, the method 400 may be performed by an information handling device 102, an analytics apparatus 104, a media module 202, an AI module 204, a score module 206, a transcription module 208, and/or a report module 210.

In one embodiment, the method 400 begins and receives 402 a recording of a conversation between a plurality of participants. In one embodiment, the method 400 transcribes the recording of the conversation to text and provide the text to the artificial intelligence engine for processing. In one embodiment, the method 400 processes 406 the recording using an artificial intelligence engine to identify at least one marker of the conversation, the at least one marker associated with at least one of the plurality of participants.

In one embodiment, the method 400 determines 408 a score for the conversation or the at least one of the plurality of participants based on the at least one marker. In one embodiment, the method 400 generates 410 a report comprising the individual scores for each of the at least one markers of the conversation for a participant of the conversation, and the method 400 ends.

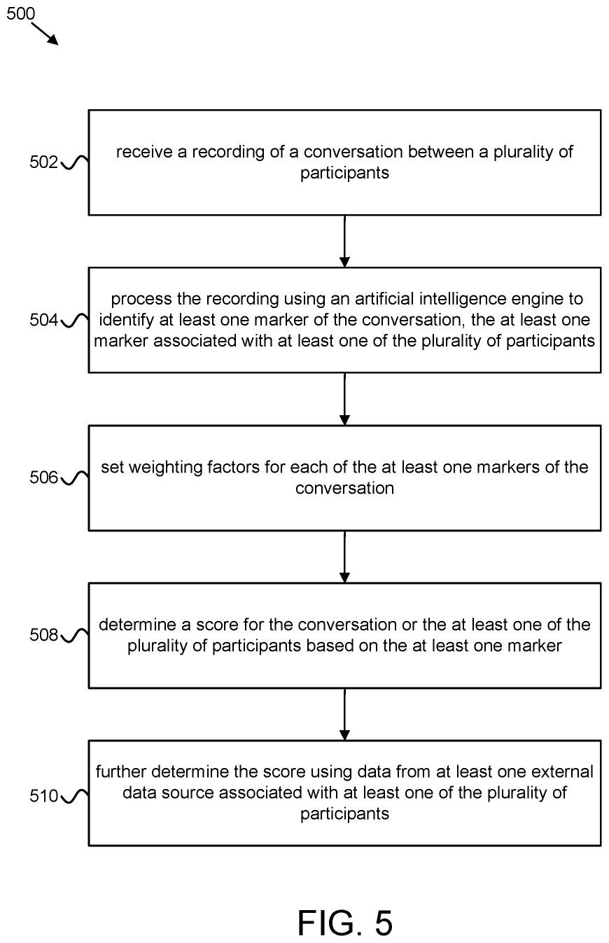

FIG. 5 depicts one embodiment of a method 500 for conversation analysis using artificial intelligence. In one embodiment, the method 500 may be performed by an information handling device 102, an analytics apparatus 104, a media module 202, an AI module 204, a score module 206, a transcription module 208, and/or a report module 210.

In one embodiment, the method 500 begins and receives 502 a recording of a conversation between a plurality of participants. In one embodiment, the method 500 processes 504 the recording using an artificial intelligence engine to identify at least one marker of the conversation, the at least one marker associated with at least one of the plurality of participants. In one embodiment, the method 500 sets 506 weighting factors for each of the at least one markers of the conversation.

In one embodiment, the method 500 determines 508 a score for the conversation or the at least one of the plurality of participants based on the at least one marker. In one embodiment, the method 500 further determines 510 the score using data from at least one external data source associated with at least one of the plurality of participants, and the method 500 ends.

An apparatus, in one embodiment, is configured to receive a recording of a conversation between a plurality of participants, process the recording using an artificial intelligence engine to identify at least one marker of the conversation, the at least one marker associated with at least one of the plurality of participants, and determine a score for the conversation or the at least one of the plurality of participants based on the at least one marker.

In one embodiment, the at least one processor is configured to cause the apparatus to transcribe the recording of the conversation to text and provide the text to the artificial intelligence engine for processing.

In one embodiment, the artificial intelligence engine is trained to identify the at least one marker of the conversation using marker-related data. In one embodiment, the at least one marker comprises a communication skill marker that indicates a communication skill of at least one of the plurality of participants.

In one embodiment, the at least one marker comprises a content marker that indicates conversational content associated with at least one of the plurality of participants. In one embodiment, the at least one marker comprises a sentiment marker that indicates emotions associated with at least one of the plurality of participants during the conversation.

In one embodiment, the at least one marker comprises an identity marker that indicates one or more identity characteristics of at least one of the plurality of participants. In one embodiment, the at least one processor is configured to cause the apparatus to set weighting factors for each of the at least one markers of the conversation, the weighting factors defining an importance of each of the at least one markers, the score determined as a function of the weighting factors.

In one embodiment, the at least one of the plurality of users is a health care provider and another of the plurality of users is a patient of the health care provider, the score of the conversation indicative of a relationship between the health care provider and the patient.

In one embodiment, the score maps to a risk factor associated with the health care provider, the risk factor indicating a level of risk associated with the health care provider. In one embodiment, the score maps to a performance level associated with the health care provider, the performance level indicating an effectiveness of the health care provider.

In one embodiment, the recording comprises a video recording of the conversation between a plurality of participants. In one embodiment, the artificial intelligence engine is configured to process the video recording for at least one visual marker.

In one embodiment, the at least one processor is configured to cause the apparatus to determine the score based on individual scores for each of the at least one markers. In one embodiment, the at least one processor is configured to cause the apparatus to generate a report comprising the individual scores for each of the at least one markers of the conversation for a participant of the conversation.

In one embodiment, the at least one processor is configured to cause the apparatus to further determine the score using data from at least one external data source associated with at least one of the plurality of participants. In one embodiment, the at least one external data source comprises survey data, note data, legal data, or a combination thereof.

In one embodiment, the apparatus is a hardware device that is specially configured for capturing the conversation between the plurality of participants and performing the functions of the apparatus.

A method, in one embodiment, includes receiving a recording of a conversation between a plurality of participants, processing the recording using an artificial intelligence engine to identify at least one marker of the conversation, the at least one marker associated with at least one of the plurality of participants, and determining a score for the conversation or the at least one of the plurality of participants based on the at least one marker.

An apparatus, in one embodiment, includes means for receiving a recording of a conversation between a plurality of participants, means for processing the recording using an artificial intelligence engine to identify at least one marker of the conversation, the at least one marker associated with at least one of the plurality of participants, and means for determining a score for the conversation or the at least one of the plurality of participants based on the at least one marker.

A means for receiving a recording of a conversation between a plurality of participants, in various embodiments, may include one or more of an information handling device 102, a server 110, an analytics apparatus 104, a media module 202, a processor (e.g., a central processing unit (CPU), a processor core, a field programmable gate array (FPGA) or other programmable logic, an application specific integrated circuit (ASIC), a controller, a microcontroller, and/or another semiconductor integrated circuit device), a hardware appliance or other hardware computing device, other logic hardware, an application, and/or other executable code stored on a computer readable storage medium. Other embodiments may include similar or equivalent means for receiving a recording of a conversation between a plurality of participants.

A means for processing the recording using an artificial intelligence engine to identify at least one marker of the conversation, in various embodiments, may include one or more of an information handling device 102, a server 110, an analytics apparatus 104, an AI module 204, a processor (e.g., a CPU, a processor core, an FPGA or other programmable logic, an ASIC, a controller, a microcontroller, and/or another semiconductor integrated circuit device), a hardware appliance or other hardware computing device, other logic hardware, an application, and/or other executable code stored on a computer readable storage medium. Other embodiments may include similar or equivalent means for processing the recording using an artificial intelligence engine to identify at least one marker of the conversation.

A means for determining a score for the conversation or the at least one of the plurality of participants based on the at least one marker, in various embodiments, may include one or more of an information handling device 102, a server 110, an analytics apparatus 104, a score module 206, a processor (e.g., a CPU, a processor core, an FPGA or other programmable logic, an ASIC, a controller, a microcontroller, and/or another semiconductor integrated circuit device), a hardware appliance or other hardware computing device, other logic hardware, an application, and/or other executable code stored on a computer readable storage medium. Other embodiments may include similar or equivalent means for determining a score for the conversation or the at least one of the plurality of participants based on the at least one marker.

A means for transcribing the recording of the conversation to text and provide the text to the artificial intelligence engine for processing, in various embodiments, may include one or more of an information handling device 102, a server 110, an analytics apparatus 104, a transcription module 208, a processor (e.g., a CPU, a processor core, an FPGA or other programmable logic, an ASIC, a controller, a microcontroller, and/or another semiconductor integrated circuit device), a hardware appliance or other hardware computing device, other logic hardware, an application, and/or other executable code stored on a computer readable storage medium. Other embodiments may include similar or equivalent means for transcribing the recording of the conversation to text and provide the text to the artificial intelligence engine for processing.

A means for generating a report comprising the individual scores for each of the at least one markers of the conversation for a participant of the conversation, in various embodiments, may include one or more of an information handling device 102, a server 110, an analytics apparatus 104, a report module 210, a processor (e.g., a CPU, a processor core, an FPGA or other programmable logic, an ASIC, a controller, a microcontroller, and/or another semiconductor integrated circuit device), a hardware appliance or other hardware computing device, other logic hardware, an application, and/or other executable code stored on a computer readable storage medium. Other embodiments may include similar or equivalent means for generating a report comprising the individual scores for each of the at least one markers of the conversation for a participant of the conversation.

Means for performing other functions described herein, in various embodiments, may include one or more of an information handling device 102, a server 110, an analytics apparatus 104, a media module 202, an AI module 204, a score module 206, a transcription module 208, a report module 210, a processor (e.g., a CPU, a processor core, an FPGA or other programmable logic, an ASIC, a controller, a microcontroller, and/or another semiconductor integrated circuit device), a hardware appliance or other hardware computing device, other logic hardware, an application, and/or other executable code stored on a computer readable storage medium. Other embodiments may include similar or equivalent means for performing other functions described herein.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, to more particularly emphasize their implementation independence. For example, a module may be implemented as a hardware circuit comprising custom very large scale integrated (“VLSI”) circuits or gate arrays, off-the-shelf semiconductors such as logic chips, transistors, or other discrete components. A module may also be implemented in programmable hardware devices such as a field programmable gate array (“FPGA”), programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (“LAN”) or a wide area network (“WAN”), or the connection may be made to an external computer (for example, through the Internet using an Internet Service Provider). In some embodiments, electronic circuitry including, for example, programmable logic circuitry, field-programmable gate arrays (“FPGA”), or programmable logic arrays (“PLA”) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program instructions may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

The schematic flowchart diagrams and/or schematic block diagrams in the Figures illustrate the architecture, functionality, and operation of possible implementations of apparatuses, systems, methods and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C. As used herein, “a member selected from the group consisting of A, B, and C and combinations thereof” includes only A, only B, only C, a combination of A and B, a combination of B and C, a combination of A and C or a combination of A, B and C.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus, comprising:
at least one memory; and
at least one processor coupled with the memory and configured to cause the apparatus to:
receive a recording of a conversation between a plurality of participants;
process the recording using an artificial intelligence engine to identify at least one marker of the conversation, the at least one marker associated with at least one of the plurality of participants; and
determine a score for the conversation or the at least one of the plurality of participants based on the at least one marker.

2. The apparatus of claim 1, wherein the at least one processor is configured to cause the apparatus to transcribe the recording of the conversation to text and provide the text to the artificial intelligence engine for processing.

3. The apparatus of claim 1, wherein the artificial intelligence engine is trained to identify the at least one marker of the conversation using marker-related data.

4. The apparatus of claim 1, wherein the at least one marker comprises a communication skill marker that indicates a communication skill of at least one of the plurality of participants.

5. The apparatus of claim 1, wherein the at least one marker comprises a content marker that indicates conversational content associated with at least one of the plurality of participants.

6. The apparatus of claim 1, wherein the at least one marker comprises a sentiment marker that indicates emotions associated with at least one of the plurality of participants during the conversation.

7. The apparatus of claim 1, wherein the at least one marker comprises an identity marker that indicates one or more identity characteristics of at least one of the plurality of participants.

8. The apparatus of claim 1, wherein the at least one processor is configured to cause the apparatus to set weighting factors for each of the at least one markers of the conversation, the weighting factors defining an importance of each of the at least one markers, the score determined as a function of the weighting factors.

9. The apparatus of claim 1, wherein at least one of the plurality of participants is a health care provider and another of the plurality of participants is a patient of the health care provider, the score of the conversation indicative of a relationship between the health care provider and the patient.

10. The apparatus of claim 9, wherein the score maps to a risk factor associated with the health care provider, the risk factor indicating a level of risk associated with the health care provider.

11. The apparatus of claim 9, wherein the score maps to a performance level associated with the health care provider, the performance level indicating an effectiveness of the health care provider.

12. The apparatus of claim 1, wherein the recording comprises a video recording of the conversation between a plurality of participants.

13. The apparatus of claim 12, wherein the artificial intelligence engine is configured to process the video recording for at least one visual marker.

14. The apparatus of claim 1, wherein the at least one processor is configured to cause the apparatus to determine the score based on individual scores for each of the at least one markers.

15. The apparatus of claim 14, wherein the at least one processor is configured to cause the apparatus to generate a report comprising the individual scores for each of the at least one markers of the conversation for a participant of the conversation.

16. The apparatus of claim 1, wherein the at least one processor is configured to cause the apparatus to further determine the score using data from at least one external data source associated with at least one of the plurality of participants.

17. The apparatus of claim 16, wherein the at least one external data source comprises survey data, note data, legal data, or a combination thereof.

18. The apparatus of claim 1, wherein the apparatus is a hardware device that is specially configured for capturing the conversation between the plurality of participants and performing functions of the apparatus.

19. A method, comprising:
receiving a recording of a conversation between a plurality of participants;
processing the recording using an artificial intelligence engine to identify at least one marker of the conversation, the at least one marker associated with at least one of the plurality of participants; and
determining a score for the conversation or the at least one of the plurality of participants based on the at least one marker.

20. An apparatus, comprising:
means for receiving a recording of a conversation between a plurality of participants;
means for processing the recording using an artificial intelligence engine to identify at least one marker of the conversation, the at least one marker associated with at least one of the plurality of participants; and
means for determining a score for the conversation or the at least one of the plurality of participants based on the at least one marker.
